(19) United States
(12) Patent Application Publication
(10) Pub. No.: US 2005/0260624 A1
(43) Pub. Date: Nov. 24, 2005

(54) Novel nucleic acid complexes and detection thereof

(76) Inventor: Chang Ning J. Wang, Andover, MA (US)

Correspondence Address: FISH & RICHARDSON PC, P.O. BOX 1022, MINNEAPOLIS, MN, 55440-1022, US

(21) Appl. No.: 11/069,370

(22) Filed: Feb. 28, 2005

Related U.S. Application Data
(60) Provisional application No. 60/548,963, filed on Feb. 28, 2004.

(51) Int. Cl. C12Q 1/68; C07H 21/04

(52) U.S. Cl. 435/6; 536/25.32

(57) ABSTRACT

A nucleic acid complex that contains a plurality of first nucleic acids, a plurality of second nucleic acids, and a plurality of third nucleic acids, in which each of the first nucleic acids, complementary to each of the second nucleic acids, contains a sequence which is complementary to a site of each of the third nucleic acids; the number of the first nucleic acids and that of the second nucleic acids are each 1 to 10¹² times that of the third nucleic acids; and the first, second, and third nucleic acids are crosslinked to form the nucleic acid complex. Also disclosed is a detection method in which the above-described nucleic acid complex is used as a detectable means for identifying a specific nucleic acid target site.

### RELATED APPLICATION

This application claims priority to U.S. Provisional Application Ser. No. 60/548,963, filed on Feb. 28, 2004, the content of which is incorporated by reference in its entirety.

### BACKGROUND

Probe hybridization has been used to detect small amounts of specific nucleic acid sequences. This detection method is not very sensitive, as it often fails to distinguish true signals from noises resulting from non-specific binding. More recently developed methods address this problem by amplifying target sequences.

Target amplification improves detection sensitivity by repeated de novo synthesis of a specific target sequence. See, e.g., U.S. Pat. Nos. 4,683,195, 4,683,202, 4,800,159, 5,455,166, 5,288,611, 5,639,604, 5,658,737, and 5,854,033; EP No. 320,308A2; International Application PCT/US87/00880; and Zehbe et al., 20 Cell Vision, vol. 1, No. 1, 1994. However, these methods are laborious and requires use of expensive instruments or enzymes. Further, their success hinges on the integrity of the target sequence. A damaged target sequence cannot be detected by these methods.

Detection sensitivity can also be enhanced by amplifying signals, cycling targets, cycling probes, or using branched DNA molecules as a signal generator. See, e.g., U.S. Pat. Nos. 4,699,876, 6,114,117, and 5,118,605; and Bekkaoui, et al., BioTechniques, 20: 240-248, 1996. Still, these methods have limited diagnostic applications due to indiscriminate amplification of background noises intrinsic to nucleic acid hybridization.

Recombinase recA-based homologous DNA strand exchange and D-loop formation have been utilized to enrich and detect nucleic acids. See, e.g., U.S. Pat. Nos. 5,670,316 and 6,335,164. These methods, nonetheless, are susceptible to interference by heterologous DNA.

There is a need to develop a nucleic acid detection method that is more sensitive, specific, and inexpensive than currently available methods.

### SUMMARY

The present invention is based on the discovery of a crosslinked nucleic acid complex that can be formed only if three of its constituent nucleic acids containing sequences homologous to one another. Of note, a very small number of copies of a nucleic acid sequence of interest is sufficient to initiate formation of the complex. Also, the complex has highly enhanced fluorescence emission after it is stained with a fluorescent dye. As a result, the sequence of interest is readily detectable at very low quantity. Furthermore, the formation of the complex can be elicited by fragmented pieces of the sequence of interest and, therefore, the detection sensitivity does not rely on sequence integrity.

Thus, this invention relates to a process for preparing a crosslinked nucleic acid complex by using a plurality of first and second nucleic acids in the presence of trace amount of third nucleic acids. The term “plurality” as used herein refers to a number of at least 10² (e.g., 10⁶). The term “trace” refers to a number of at least 1. Each of the first nucleic acids is complementary to each of the second nucleic acids, and contains a sequence that is complementary to a site, i.e., a segment, of each of the third nucleic acids. The first nucleic acids and the second nucleic acids can be conveniently provided as a double stranded DNA and their numbers are each 1 to 1016 times (preferably, 103 to 10¹³ times) that of the third nucleic acids. They each have a length of 100 to 20,000 nucleotides (e.g., 200 to 8,000 nucleotides). The complementary sequence between a first nucleic acid and a third nucleic acid can have a length of 10 to 20,000 nucleotides (e.g., 20 to 8,000 nucleotides). To facilitate formation of a crosslinked nucleic acid complex, one denatures the first, second, and third nucleic acids, and localizes them to a planar surface. As an example, one can use a chaotropic aqueous solvent for denaturing the nucleic acids, and then add a hydrophobic organic solvent to the chaotropic aqueous solvent for localizing the nucleic acids at the interface between the two solvents. The crosslinked nucleic acid complex thus formed can further be extracted from the mixture of the two solvents, if necessary.

In the crosslinked nucleic acid complex thus obtained, which is within the scope of this invention, the number of the first or second nucleic acids is 1 to 10¹² times (preferably, 10³ to 10⁸ times) that of the third nucleic acids. The complex possesses an unusual fluorescence property. When excited at 518 nm after staining with ethidium bromide, it emits fluorescence at 605 nm with an intensity at least 10 times that of ethidium bromide-stained non-crosslinked first, second, and third nucleic acids.

The crosslinked nucleic acid complex can serve as a detectable means in a method for identifying a target site in a nucleic acid sequence, e.g., obtained from a sample. More specifically, when the third nucleic acids (mentioned above) are DNA or RNA molecules suspected of containing a target site complementary to a sequence of each of the first nucleic acids (also mentioned above), identification of the target site can be achieved by conducting the above-described process. Since a crosslinked nucleic acid complex does not form in the absence of the target site, detection of such a complex indicates that the target site is present. The formation of the crosslinked nucleic acid complex can be verified by the rise of its apparent molecular weight and its quantity can be determined by its fluorescence emission intensity after staining with a double stranded DNA intercalating fluorescent dye.

Of note, sequence integrity of the third nucleic acids does not affect detection as de novo DNA synthesis is not required for the complex formation. This advantage enables detection of even damaged DNA, i.e., less than one intact target site, and cannot be achieved by PCR-based amplification methods.

Also within the scope of this invention is a multiphasic system in which the above-described process can be conducted to form a crosslinked nucleic acid complex. This multiphasic system includes a hydrophobic organic solvent and a chaotropic aqueous solvent, separated from each other into two phases at mixing, and a plurality of nucleic acids at the planar interfacial surface between the two solvents. The nucleic acids at the planar interfacial surface can be a mixture of the first, second, and third nucleic acids (mentioned above), a mixture of the first and second nucleic acids, or that of the third nucleic acids, depending on the order in which the three nucleic acids are introduced to a mixture of the hydrophobic organic solvent and the chaotropic aqueous solvent.

Other features, objects, and advantages of the invention will be apparent from the description and from the claims.

### DETAILED DESCRIPTION

To demonstrate how a nucleic acid complex of this invention can be prepared, described in detail below is formation of such a complex in a process for detecting a target nucleic acid site.

The target site can be a part of a single stranded nucleic acid (e.g., a single stranded viral DNA or a human mRNA), a double stranded DNA (e.g., a double stranded viral DNA or a human genomic DNA), a DNA-RNA hybrid, and combinations of one or more of the above. For example, a double stranded viral DNA isolated from human blood can be detected as follows. One first selects a target site to be detected by identifying a consensus sequence of the viral genome that is unique to the virus and absent from the human and other viral DNAs. One then constructs by standard molecular cloning techniques a double stranded DNA probe containing a sequence complementary to the selected target site. The double stranded viral DNA and the double stranded probe (preferably, 10³ to 10¹⁰ times the number of copies of the viral DNA) are then denatured in a chaotropic aqueous solvent, in which the viral DNA and the probe are destabilized and their respective complementary strands dissociate to adopt an unwound conformation. A hydrophobic organic solvent is then added to the aqueous solvent to create a biphasic system, in which a planar surface is formed between the two solvents. Examples of suitable hydrophobic organic solvents include aniline, n-butylalcohol, tert-amylalcohol, cyclohexyl alcohol, phenol, p-methoxyphenol, benzyl alcohol, pyridine, purine, 3-aminotriazole, butyramide, hexamide, thioacetamide, δ-valarolactam, tert-butylurea, ethylenethiourea, allylthiourea, thiourea, urethane, N-propylurethane, N-methylurethane, cyanoguanidine, and combinations of two or more of the above. Examples of suitable chaotropic aqueous solvents include those containing one or more of SCN⁻, Mg²⁺, Ca²⁺, Na⁺, K⁺, NH₄⁺, Cs⁺, Li⁺, and (CH₃)₄N⁺, in combination with those containing one or more of tosylate⁻, Cl₃CCOO⁻, ClO₄⁻, I⁻, Br⁻, Cl⁻, BrO₃⁻, CH₃COO⁻, HSO₃⁻, F⁻, SO₄²⁻, (CH₃)₃CCOO⁻, and HPO₄⁻.

Described below is a postulated mechanism by which a crosslinked nucleic acid complex is formed. The double stranded probe and the double stranded viral DNA in a chaotropic aqueous solvent-hydrophobic organic solvent system (i.e., an amphipathic environment) will be attracted to the interfacial surface between the organic and aqueous phases and expose their hydrophilic phosphoribose moieties to the aqueous phase and their its hydrophobic ring moieties to the organic phase. In order word, Watson-Crick pairing cannot be maintained. Further, the two complementary probe strands (as well as the two complementary strands of the viral DNA) thus stabilized pair side-by-side in close proximity to each other on the same plane, i.e., in paranemic pairing. Regional pairing between a pair of paranemic probe strands is disrupted by a target site-containing viral DNA strand that comes to pair with the probe strand that contains a sequence complementary to the target site. Beyond the disrupted region, the probe strands remain engaged in paranemic pairing. In other words, the complementary probe strands are only partially displaced. Yet, a kink is created at either end of the disrupted region. The kink distorts the topological structure of the paranemic probe strands and forces parts of them from the planar surface into the aqueous phase. On the planar surface, the partially displaced probe strand that contains a sequence identical to the target site further pairs side-by-side with a member of another pair of paranemic probe strands, leaving behind the other member of that pair partially displaced and ready for pairing with a member of still another pair of the paranemic probe strands. Under controlled conditions, this process continues until the complex formation process is no longer energetically favorable as the probe strands are depleted and prevented from further paranemic pairing. As a result, a small number of copies of the viral DNA is sufficient to trigger a cascade of crosslinking events among the probe strands, generating a crosslinked nucleic acid complex. The complex thus formed can be easily isolated by ethanol or isopropanol precipitation in the presence of a chaotropic aqueous solution.

The crosslinked complex thus obtained, when excited at 518 nm after staining with ethidium bromide, emits fluorescence at 605 nm with an intensity at least 10 times that of ethidium bromide-stained non-crosslinked probe nucleic acid and viral DNA. In essence, this much enhanced fluorescence intensity can be determined as follows. 100 ng of the crosslinked complex is stained with 0.25 μg/ml ethidium bromide for 5 minutes. The fluorescence intensity is then measured and compared with that obtained from 100 ng of the non-processed nucleic acids, i.e., the viral DNA and the double stranded probe. The complex can also be detected by other methods. For example, it can be visualized after resolving by gel electrophoresis. Presence of a crosslinked complex is indicated by a band on the gel with a molecular weight larger than the combined molecular weights of the non-processed nucleic acids. Alternatively, the complex can be detected as a species farther removed from the axis of rotation as compared to the non-processed nucleic acids during sedimentation equilibrium process. One can also detect the complex by microscopy. For example, fluorescent dye stained complex can be observed under a fluorescence microscope after moisture chamber vaporization on a microscope slide. The amount of complex formed can also be quantified by quantitative PCR (QPCR) after the unreacted probe, present in single stranded form, was removed by digestion with a single stranded DNA specific nuclease (e.g., mung bean nuclease). Target-specific primers can be used to amplify the target sequences in the presence of signal generating primer (e.g. AmpliSensor) or probe (e.g. TaqMan probe) to reveal the total amount of the probe nucleic acid engaged in the complex.

Nucleic acids from biological or other samples are preferably purified prior to the detection assay. For example, a sample (e.g., blood, lymphatic fluid, urine, food, or sewage) can be first incubated in a lysis buffer. Ethanol or isopropanol is then added to facilitate nucleic acid precipitation. As described above, the nucleic acid, as well as the double stranded probe, can be denatured by a chaotropic aqueous solvent mentioned before. The concentration of the chaotropic agent(s) can be determined empirically such that the complementary strands of the probe nucleic acids, after the denaturation, still pair with each other side by side on a planar surface.

In general, detection sensitivity can be improved by augmenting the quantity or increasing the length the probe strands. By including in the probe a high energy barrier (i.e., a higher GC content) region as a clamp to prevent branch migration, it will also stabilize the complex after it has been formed. One can also increase detection sensitivity by repeating the complex formation process after fragmenting the complex prior to each repeat. A complex can be fragmented with T7 endonuclease I, which digests mismatched DNA and Holliday structures. A fragmented complex will resume the Watson Crick base pairing of a canonical B form helix. and can thus trigger the crosslinking events with new supply of the double stranded probe.

One can use multiple probes specific to a single target site or a single probe specific to multiple target sites to detect different target sites in one single assay. Since the signal, even indiscriminative, will be directly related to amount of individual target sites present in the reaction, this approach is particularly useful for simultaneously detecting multiple pathogens in a sample.

The procedure described above can also be used to prepare coating material by replacing, if necessary, the viral DNA with any suitable nucleic acid sequence. The crosslinked complex has a high charge density due to the polyanionic groups of the nucleic acids, and can be used to coat a surface for immobilizing cationic molecules. It can be applied to a surface as a thin-film by standard spraying techniques.

Also within the scope of this invention is a kit for detecting specific nucleic acid sequences by the above-described process. The kit can contain two or more of the following reagents: a probe nucleic acid specific to a target sequence, a chaotropic reagent or a chaotropic aqueous solvent, a hydrophobic organic solvent, and a fluorescent dye for detection.

Without further elaboration, it is believed that one skilled in the art can, based on the description above (including a postulated mechanism, which does not restrict the scope of this invention as claimed), utilize the present invention to its fullest extent. All publications cited herein are hereby incorporated by reference in their entirety. The specific examples below are to be construed as merely illustrative, and not limitative of the remainder of the disclosure in any way whatsoever.

### EXAMPLES

Human hepatitis B virus (HBV) genome was detected using a nucleic acid sequence that contained a segment corresponding to the HBV surface antigen specific sequence (HBVSAg). This HBVSAg segment was PCR-amplified from a source of the HBV genome using the following primers: TCG TGG TGG ACT TCT CTC AAT TTT CTA GG, (SEQ ID NO:1) and CGA GGC ATA GCA GCA GGA TGA AGA GA (SEQ ID NO:2). The PCR-amplified HBVSAg segment was then sub-cloned into a modified PUC18 plasmid via a HincII restriction site. The HBVSAg/PUC18 plasmid thus obtained was propagated in E. coli DH5α strain. 10 μg of this plasmid, isolated by plasmid extraction from E. coli, was subsequently digested with restriction enzyme EcoRI, generating a ˜3 kb fragment that contained the HBVSAg segment. The fragment, a double stranded DNA probe, was used as a probe to detect HBV Shown below is the sequence of one of the two strands of the DNA probe. This sequence contains a “clamp” region (shown in boldface), which provides a distinct energy barrier to keep the two strands from dissociating.

| (SEQ ID NO:3) | |
|---|---|
| 1 | aattcgtaat catggtcata gctgtttcct gtgtgaaatt gttatccgct cacaattcca |
| 61 | cacaacatac gagccggaag cataaagtgt aaagcctggg gtgcctaatg agtgagctaa |
| 121 | ctcacattaa ttgcgttgcg ctcactgccc gctttccagt cgggaaacct gtcgtgccag |
| 181 | ctgcattaat gaatcggcca acgcgcgggg agaggcggtt tgcgtattgg gcgctcttcc |
| 241 | gcttcctcgc tcactgactc gctgcgctcg gtcgttcggc tgcggcgagc ggtatcagct |
| 301 | cactcaaagg cggtaatacg gttatccaca gaatcagggg ataacgcagg aaagaacatg |
| 361 | tgagcaaaag gccagcaaaa ggccaggaac cgtaaaaagg ccgcgttgct ggcgtttttc |
| 421 | cataggctcc gcccccctga cgagcatcac aaaaatcgac gctcaagtca gaggtggcga |
| 481 | aacccgacag gactataaag ataccaggcg tttccccctg gaagctccct cgtgcgctct |
| 541 | cctgttccga ccctgccgct taccggatac ctgtccgcct ttctcccttc gggaagcgtg |
| 601 | gcgctttctc atagctcacg ctgtaggtat ctcagttcgg tgtaggtcgt tcgctccaag |
| 661 | ctgggctgtg tgcacgaacc ccccgttcag cccgaccgct gcgccttatc cggtaactat |
| 721 | cgtcttgagt ccaacccggt aagacacgac ttatcgccac tggcagcagc cactggtaac |
| 781 | aggattagca gagcgaggta tgtaggcggt gctacagagt tcttgaagtg gtggcctaac |
| 841 | tacggctaca ctagaaggac agtatttggt atctgcgctc tgctgaagcc agttaccttc |
| 901 | ggaaaaagag ttggtagctc ttgatccggc aaacaaacca ccgctggtag cggtggtttt |
| 961 | tttgtttgca agcagcagat tacgcgcaga aaaaaaggat ctcaagaaga tcctttgatc |
| 1021 | ttttctacgg ggtctgacgc tcagtggaac gaaaactcac gttaagggat tttggtcatg |
| 1081 | agattatcaa aaaggatctt cacctagatc cttttaaatt aaaaatgaag ttttaaatca |
| 1141 | atctaaagta tatatgagta aacttggtct gacagttacc aatgcttaat cagtgaggca |
| 1201 | cctatctcag cgatctgtct atttcgttca tccatagttg cctgactccc cgtcgtgtag |
| 1261 | ataactacga tacgggaggg cttaccatct ggccccagtg ctgcaatgat accgcgagac |
| 1321 | ccacgctcac cggctccaga tttatcagca ataaaccagc cagccggaag ggccgagcgc |
| 1381 | agaagtggtc ctgcaacttt atccgcdtcc atccagtcta ttaattgttg ccgggaagct |
| 1441 | agagtaagta gttcgccagt taatagtttg cgcaacgttg ttgccattgc tacaggcatc |
| 1501 | gtggtgtcac gctcgtcgtt tggtatggct tcattcagct ccggttccca acgatcaagg |
| 1561 | cgagttacat gatcccccat gttgtgcaaa aaagcggtta gctccttcgg tcctccgatc |
| 1621 | gttgtcagaa gtaagttggc cgcagtgtta tcactcatgg ttatggcagc actgcataat |
| 1681 | tctcttactg tcatgccatc cgtaagatgc ttttctgtga ctggtgagta ctcaaccaag |
| 1741 | tcattctgag aatagtgtat gcggcgaccg agttgctctt gcccggcgtc aatacgggat |
| 1801 | aataccgcgc cacatagcag aactttaaaa gtgctcatca ttggaaaacg ttcttcgggg |
| 1861 | cgaaaactct caaggatctt accgctgttg agatccagtt cgatgtaacc cactcgtgca |
| 1921 | cccaactgat cttcagcatc ttttactttc accagcgttt ctgggtgagc aaaaacagga |
| 1981 | aggcaaaatg ccgcaaaaaa gggaataagg gcgacacgga aatgttgaat actcatactc |
| 2041 | ttcctttttc aatattattg aagcatttat cagggttatt gtctcatgag cggatacata |
| 2101 | tttgaatgta tttagaaaaa taaacaaata ggggttccgc gcacatttcc ccgaaaagtg |
| 2161 | ccacctgacg tctaagaaac cattattatc atgacattaa cctataaaaa taggcgtatc |
| 2221 | acgaggccct ttcgtctcgc gcgtttcggt gatgacggtg aaaacctctg acacatgcag |
| 2281 | ctcccggaga cggtcacagc ttgtctgtaa gcggatgccg ggagcagaca agcccgtcag |
| 2341 | ggcgcgtcag cgggtgttgg cgggtgtcgg ggctggctta actatgcggc atcagagcag |
| 2401 | attgtactga gagtgcacca tatgcggtgt gaaataccgc acagatgcgt aaggagaaaa |
| 2461 | taccgcatca ggcgccattc gccattcagg ctgcgcaact gttgggaagg gcgatcggtg |
| 2521 | cgggcctctt cgctattacg ccagctggcg aaagggggat gtgctgcaag gcgattaagt |
| 2581 | tgggtaacgc cagggttttc ccagtcacga cgttgtaaaa cgacggccag tgccaagctt |
| 2641 | gcatgcctgc aggtctcgtg gtggacttct ctcaattttc tagggggaac acccgtgtgt |
| 2701 | cttggccaaa attcgcagtc ccaaatctcc agtcactcac caacttgttg tcctccgatt |
| 2761 | tgtcctggtt atcgctggat gtgtctgcgg cgttttatca tctttctctt catcctgctg |
| 2821 | ctatgcctcg gactctagag gatccccggg taccgagctc g |

5×10² to 5×10⁻² copies of the HBV genome were resuspended in 20 μl of 1.0 M GuSCN and 50 mM potassium phosphate (pH 6.0). 15 ng of the probe was then added to each of the HBV genome solution, followed by 20 μl of aniline to form a biphasic system containing a chaotropic aqueous solvent and a hydrophobic organic solvent. The mixture was vortexed and incubated at 30° C. for 15 minutes to allow formation of the complex.

The complex was isolated as follows. 5 M guanidinium chloride and isopropanol were added to the above mixture. After vortexing and centrifugation at 14,000 rpm for 5 minutes, the supernatant was decanted and the pellet, which contained the complex, was washed with 75% ethanol, air-dried for 10 minutes, and resuspended in 20 μl Tris-EDTA buffer.

The complex was observed as follows. 0.2 μl of PicoGreen dsDNA Quantitation Reagent (obtained from Molecular Probe, Inc. and identified as # P-7581) was added to 10 μl of the resuspended nucleic acid complex. 5 μl of the labeled nucleic acid complex was then applied to a microscope slide (obtained from Kevley Technologies and identified as #CFR) and the slide was air-dried overnight. Aggregates of the complex were observed by microscopy at 250× magnification under both fluorescence and optical settings.

The complex was also resolved by gel electrophoresis as follows. 10 μl of the nucleic acid complex was applied to a horizontal 1% agarose gel in 0.5× TBE buffer and electrophoresed under 4V/cm for 8 hours. The gel was then stained with 0.5 μg/ml of ethidium bromide. Photographs of the gel were taken with a red filter under UV illumination. Two distinct bands were observed from the lane of the complex. Sizes of the two bands were ˜10 kb (relaxed) and ˜4 kb (compact), respectively. On the same gel, the size of the nucleic acid sequence that contained the HBVSAg segment was confirmed to be ˜2.8 kb and the HBV genome to be ˜3.2 kb. Thus, the size of the complex (i.e., ˜10 kb relaxed form) was higher than the combined sizes of the HBVSAg-containing nucleic acid sequence and the HBV genome.

Further, the complex was fragmented by digesting with T7 endonuclease I as follows. 10 μl of the complex was incubated with 2 units of T7 endonuclease I (obtained from New England BioLabs, Inc. and identified as M0292S) for 1 hour at 42° C. in 15 μl of 50 mM potassium acetate, 20 mM Tris acetate (pH 7.9), 10 mM magnesium acetate, 1 mM dithiothretol (DTT). The fragmented complex was used as a starting material for another round of complex formation.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A nucleic acid complex, comprising a plurality of first nucleic acids, a plurality of second nucleic acids, and a plurality of third nucleic acids, wherein each of the first nucleic acids, complementary to each of the second nucleic acids, contains a sequence which is complementary to a site of each of the third nucleic acids; the number of the first nucleic acids and the number of the second nucleic acids are each 1 to 10¹² times that of the third nucleic acids; and the first nucleic acids, the second nucleic acids, the third nucleic acids are crosslinked to form the nucleic acid complex, which, when excited at 518 nm after staining with ethidium bromide, emits fluorescence at 605 nm with an intensity at least 10 times that of non-crosslinked first, second, and third nucleic acids.

2. The nucleic acid complex of claim 1, wherein the number of the first nucleic acids and the number of the second nucleic acids are each 10³ to 10⁸ times that of the third nucleic acids.

3. The nucleic acid complex of claim 2, wherein the first and second nucleic acids are each 100 to 20,000 nucleotides in length.

4. The nucleic acid complex of claim 3, wherein the first and second nucleic acids are each 200 to 8,000 nucleotides in length.

5. The nucleic acid complex of claim 4, wherein the complementary sequence is 10 to 20,000 nucleotides in length.

6. The nucleic acid complex of claim 5, wherein the complementary sequence is 20 to 8,000 nucleotides in length.

7. The nucleic acid complex of claim 2, wherein the complementary sequence is 10 to 20,000 nucleotides in length.

8. The nucleic acid complex of claim 7, wherein the complementary sequence is 20 to 8,000 nucleotides in length.

9. The nucleic acid complex of claim 3, wherein the complementary sequence is 10 to 20,000 nucleotides in length.

10. The nucleic acid complex of claim 9, wherein the complementary sequence is 20 to 8,000 nucleotides in length.

11. A process for detecting a target site in a nucleic acid, comprising:
providing a plurality of first nucleic acids, a plurality of second nucleic acids, and a plurality of third nucleic acids suspected of containing a target site, wherein each of the first nucleic acids, complementary to each of the second nucleic acids, contains a sequence which is complementary to a target site of each of the third nucleic acids; and the number of the first nucleic acids and the number of the second nucleic acids are each 1 to 10¹⁶ times that of the third nucleic acids;
denaturing and localizing to a planar surface the first, second, and third nucleic acids, thereby facilitating formation of a crosslinked nucleic acid complex if each of the third nucleic acids contains the target site; and
detecting presence or absence of the crosslinked nucleic acid complex.

12. The process of claim 11, wherein the number of the first nucleic acids and the number of the second nucleic acids are each 10³ to 10¹³ times that of the third nucleic acids.

13. The process of claim 12, wherein the first and second nucleic acids are each 100 to 20,000 nucleotides in length.

14. The process of claim 13, wherein the first and second nucleic acids are each 200 to 8,000 nucleotides in length.

15. The process of claim 14, wherein the complementary sequence is 10 to 20,000 nucleotides in length.

16. The process of claim 15, wherein the complementary sequence is 20 to 8,000 nucleotides in length.

17. The process of claim 16, wherein the denaturation is achieved by mixing the first, second, and third nucleic acids in a chaotropic aqueous solvent.

18. The process of claim 17, wherein the localization is achieved by adding a hydrophobic organic solvent to the chaotropic aqueous solvent, the interface between the two solvents constituting the planar surface.

19. The process of claim 18, wherein the hydrophobic organic solvent is n-butylalcohol, tert-amylalcohol, cyclohexyl alcohol, phenol, p-methoxyphenol, benzyl alcohol, aniline, pyridine, purine, 3-aminotriazole, butyramide, hexamide, thioacetamide, δ-valarolactam, tert-butylurea, ethylenethiourea, allylthiourea, thiourea, urethane, N-propylurethane, N-methylurethane, cyanoguanidine, or a combination thereof.

20. The process of claim 19, wherein the hydrophobic organic solvent is aniline.

21. The process of claim 18, wherein the chaotropic aqueous solvent contains Mg²⁺, Ca²⁺, Na⁺, K⁺, NH₄⁺, Cs⁺, Li⁺, (CH₃)₄N⁺, or a combination thereof.

22. The process of claim 18, wherein the chaotropic aqueous solvent contains tosylate⁻, Cl₃CCOO⁻, SCN⁻, ClO₄⁻, I⁻, Br⁻, Cl⁻, BrO₃⁻, CH₃COO⁻, HSO₃⁻, F⁻, SO₄²⁻, (CH₃) ₃CCOO⁻, HPO₄⁻, or a combination thereof.

23. The process of claim 22, wherein the chaotropic aqueous solvent contains SCN⁻.

24. The process of claim 12, wherein the denaturation is achieved by mixing the first, second, and third nucleic acids in a chaotropic aqueous solvent.

25. The process of claim 24, wherein the localization is achieved by adding a hydrophobic organic solvent to the chaotropic aqueous solvent, the interface between the two solvents constituting the planar surface.

26. The process of claim 25, wherein the detection is achieved by determining the intensity of fluorescence emitted from the crosslinked nucleic acid complex after it is stained with a double stranded DNA intercalating fluorescent dye.


SEQUENCE LISTING (3 sequences)

SEQ ID NO: 1 - DNA, 29 bp, Artificial Sequence
  Primer
tcgtggtggacttctctcaattttctagg

SEQ ID NO: 2 - DNA, 26 bp, Artificial Sequence
  Primer
cgaggcatagcagcaggatgaagaga

SEQ ID NO: 3 - DNA, 2861 bp, Artificial Sequence
  Synthetic construct
aattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtctcgtggtggacttctctcaattttctagggggaacacccgtgtgtcttggccaaaattcgcagtcccaaatctccagtcactcaccaacttgttgtcctccgatttgtcctggttatcgctggatgtgtctgcggcgttttatcatctttctcttcatcctgctgctatgcctcggactctagaggatccccgggtaccgagctcg